(19) United States
(12) Patent Application Publication
(10) Pub. No.: US 2009/0104251 A1
(43) Pub. Date: Apr. 23, 2009

(54) HEAT STABLE MICROCAPSULES AND METHODS FOR MAKING AND USING THE SAME

(75) Inventor: Phillip K. Lee, Avon, IN (US)

Correspondence Address: MICHAEL BEST & FRIEDRICH LLP, 100 E WISCONSIN AVENUE, Suite 3300, MILWAUKEE, WI, 53202, US

(73) Assignee: SENSIENT FLAVORS INC., Indianapolis, IN (US)

(21) Appl. No.: 11/876,247

(22) Filed: Oct. 22, 2007

(51) Int. Cl. A61K 9/50; A61K 47/00

(52) U.S. Cl. 424/439; 424/490

(57) ABSTRACT

A method of manufacturing a microcapsule that may include complex coacervating a protein and a polyanionic polymer to form a coacervate, the coacervate being at least a portion of the microcapsule, and wherein a cross-linking reagent is not used during the coacervation.

### FIELD OF USE

The present invention relates to heat stable microcapsule compositions, and particularly, their use in food and pharmaceutical products. The invention also relates to methods of making microcapsule compositions.

### BACKGROUND

Over the years, considerable effort has been expended to encapsulate active ingredients, such as nutrients, and include them in various food and pharmaceutical products. However, these encapsulates are often unstable and can possess offensive flavors and odors. Microcapsules that are heat stable and that may include active ingredients while optionally masking unpleasant tastes and odors are desirable.

### SUMMARY

In one aspect, a method of manufacturing a microcapsule is provided, where the method may include complex coacervating a protein and a polyanionic polymer to form a coacervate, the coacervate being at least a portion of the microcapsule, and wherein a cross-linking reagent is not used during the coacervation.

In another aspect, a microcapsule composition that may include a protein, a polyanionic polymer, and a taste masking agent is provided.

In yet another aspect, an encapsulate that may include a protein and gellan gum is provided.

In a further aspect, a microcapsule composition that may include an encapsulate including a protein, a polyanionic polymer, and an active ingredient encapsulated in the encapsulate is provided. When the microcapsule composition is exposed to retort processing at 121° C. and 15 PSI for 60 minutes, or hot fill pasteurization at 104° C., substantially all of the active ingredient remains in the encapsulate.

In another aspect, a method of delivering an active ingredient to a subject is provided. The method may include incorporating a microcapsule composition including a protein, a polyanionic polymer, an active ingredient, and a taste masking agent into at least one of a food, a beverage, a pet food, a pharmaceutical, a drug delivery system, and a combination thereof; and administering at least one of a food, a beverage, a pet food, a pharmaceutical, a drug delivery system, and a combination thereof to the subject.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

In one embodiment, the invention may provide a microcapsule composition comprising at least one of a protein and a polyanionic polymer. The microcapsule composition may optionally contain active ingredients, cross-linking reagents, additional taste masking agents, or combinations thereof. The microcapsule compositions may be suitably edible and non-toxic.

Examples of proteins include, but are not limited to, soy protein isolate, milk protein, canola protein, whey protein, gelatin (bovine), gelatin (porcine), gelatin (fish), albumin, or combinations thereof. Examples of soy protein isolates include, but are not limited to, Supro EX 38, Supro SP 248, and Supro XT 219 (all available from Solae, St. Louis, Mo.).

Examples of polyanionic polymers include, but are not limited to, polycarboxylated polymers, polyaspartic acid or salts thereof, polyglutamic acid or salts thereof, agars, alginate, starches, gum Arabic, carrageenan, pectin, hydroxypropylmethyl cellulose, gellan gum, or combinations thereof.

Active ingredients include any materials or substances that may be useful in the present invention. For example, active ingredients may include, but are not limited to, pharmaceutically active drugs, catalysts, living or dead cells, tissue, agriculturally useful substances such as pesticides, herbicides, nutrients, and fertilizers or seeds, aquaculturally useful substances such as feeds or pigments, cosmetic products, particulates, food ingredients, and combinations thereof. In some embodiments, the microcapsules need not include any active ingredient at all, or may include only additives providing a desired color or taste, or sweeteners or the like.

Examples of specific food ingredients may include, but are not limited to, greases, oils, lipids, drugs, nutritional supplements such as vitamins, flavor compounds, carotenoids such as lycopene, satiety agents, drugs, antioxidants, or combinations thereof. Examples of oils may include, but are not limited to, animal oils, vegetable oils, mineral oils, derivatives thereof or combinations thereof. Examples of specific animal oils may include, but are not limited to, fish oils or marine mammal oils. Examples of fish oils may include, but are not limited to, alkaline treated fish oil, anchovy oil, Atlantic fish oils, Atlantic cod oil, Atlantic herring oil, Atlantic mackerel oil, Atlantic menhaden oil, barracuda oil, capelin oil, cod oil, halibut oil, heat treated fish oil, light and heavy brown fish oil, light pressed fish oil, Mediterranean fish oils, menhaden oil, Pacific fish oils, salmonid oil, sardine oil, sea bass oil, shark oil, spearfish oil, tuna oil, or combinations thereof. Examples of specific antioxidants include, but are not limited to, vitamin E, CoQ₁₀, tocopherols, plant extracts such as rosemary, sage, and oregano oils, lipid soluble derivatives of more polar antioxidants such as ascobyl fatty acid esters (for example, ascobyl palmitate), algal extracts, synthetic antioxidants such as BHT, TBHQ, ethoxyquin, alkyl gallates, hydroquinones, and tocotrienols, or combinations thereof.

The active ingredient may include purified or partially purified oily substances such as fatty acids, triglycerides or esters thereof, or combinations thereof. Examples of fatty acids may include, but are not limited to, fatty acids comprising at least 10, at least 12, at least 14, at least 16, at least 18, or at least 20 carbon atoms. Specific examples of fatty acids include those containing anywhere from 10 to 45 carbon atoms. In other examples, the fatty acids can comprise a range of carbon atoms. For example, the fatty acids can comprise from about 6 to about 40, from about 12 to about 38, from about 14 to about 36, from about 16 to about 34, from about 18 to about 32, or from about 20 to about 30 carbon atoms. The fatty acids can be unsaturated, saturated, or a combination thereof. Specific examples include, but are not limited to, omega-3 fatty acids. Examples of omega-3 fatty acids include, but are not limited to, α-linolenic acid (18:3ω3), docosahexaenoic acid (22:6ω3) (DHA), docosapentaenoic acid (22:5ω3) (DPA), eicosapentaenoic acid (20:5ω3) (EPA), eicosatetraenoic acid (20:4ω3), octadecatetraenoic acid (18:4ω3), uncosapentaenoic acid (21:5ω3) and derivatives thereof and combinations thereof. Examples of suitable derivatives include, but are not limited to, esters, such as phytosterol esters, branched or unbranched C₁-C₃₀ alkyl esters, branched or unbranched C₂-C₃₀ alkenyl esters, or branched or unbranched C₃-C₃₀ cycloalkyl esters such as phytosterol esters and C₁-C₆ alkyl esters. Sources of oils can be derived from, without limitation, aquatic organisms such as anchovies, Atlantic cod, Atlantic herring, Atlantic mackerel, Atlantic menhaden, capelin, salmonids, sardines, shark, tuna, etc., plants such as flax, vegetables, etc., microorganisms such as fungi and algae, or combinations thereof.

The microcapsule composition of the present invention does not require cross-linking reagents. This may result in a more efficient method of manufacturing the microcapsules, which can lead to cost savings. However, cross-linking reagents may be used. Any suitable cross-linking reagent may be used, depending on the choice of polymer and protein. Cross-linking reagents are suitably non-toxic or of suitably low toxicity for food, beverage, and pharmaceutical applications. Examples of specific cross-linking reagents include, but are not limited to, enzymatic cross-linkers such as transglutaminase, chemical cross-linkers such as aldehydes such as formaldehyde or glutaraldehyde, tannic acid, alum, or combinations thereof.

The microcapsule compositions of the present invention may further comprise taste masking agents. This secondary taste masking technology may be separate from the coating itself. A tiny amount of residual active ingredient can be found on the outside of the microcapsules and can cause flavor and odor problems. Taste masking agents targeted to specific food applications may be co-encapsulated to reduce the adverse flavor impact from the coating polymers or active ingredients. Examples of taste masking agents may include, but are not limited to, food grade flavors. The food grade flavors may be synthetic or artificial flavors, natural flavors or any mixture thereof. Examples of suitable flavors include, but are not limited to, almond, amaretto, apple, green apple, apple-cherry-berry, apple-honey, apricot, bacon, balls of fire, banana, barbeque, bay, beef, roast beef, beef steak, berry, berry blue, birch beer/spruce beer, blackberry, bloody mary, blueberry, boysenberry, brandy, bubble gum, butter, butter pecan, buttermilk, butterscotch, candy corn, cantaloupe, cantaloupe lime, caramel, carrot, cassia, caviar, celery, cereal, champagne, cherry, cherry cola, cherry maraschino, wild cherry, black cherry, red cherry, cherry-cola, chicken, chocolate, chocolate almond, cinnamon spice, citrus, citrus blend, citrus-strawberry, clam, cocoa, coconut, toasted coconut, coffee, coffee almond, cola, cola-vanilla, cookies & cream, cool, cotton candy, cranberry, cranberry-raspberry, cream, cream soda, dairy type cream, crème de menthe, cucumber, black currant, dulce de leche, egg nog, pork fat, type fat, anchovy fish, herring fish, sardine fish, frankfurter, fiery hot, fried garlic, sautéed garlic, gin, ginger ale, ginger beer, graham cracker type, grape, grape grapefruit, grapefruit-lemon, grapefruit-lime, grenadine, grill, guarana, guava, hazelnut, honey, hot, roasted honey, ice cream cone, jalapeno, key lime, kiwi, kiwi-banana, kiwi-lemon-lime, kiwi-strawberry, kola champagne, lard type, lemon, lemon custard, lemonade, pink lemonade, lemon-lime, lime, malt, malted milk, mango, mango-pineapple, maple, margarita, marshmallow, meat type, condensed milk, cooked milk, mint, mirepoix, mocha, mochacinna, molasses, mushroom, sautéed mushroom, muskmelon, nectarine, neopolitan, green onion, sautéed onion, orange, orange cordial, orange creamsicle, orange crème, orange peach mango, orange strawberry banana, creamy orange, mandarin orange, orange-passion-guava, orange-pineapple, papaya, passion fruit, peach, peach-mango, peanut, roasted peanut, pear, pecan danish type, pecan praline, pepper, peppermint, pimento, pina colada, pina colada/pineapple-coconut, pineapple, pineapple-orange, pistachio, pizza, pomegranate, pork fat type, baked potato, prune, punch, citrus punch, tropical punch, cherry fruit punch, grape punch, raspberry, black raspberry, blue raspberry, red raspberry, raspberry-blackberry, raspberry-ginger ale, raspberry-lime, roast type, root beer, rum, sangria, sarsaparilla, sassafras, sausage, sausage pizza, savory, seafood, shrimp, hickory smoke, mesquite smoke, sour, sour cream, sour cream and onion, spearmint, spicy, strawberry, strawberry margarita, jam type strawberry, strawberry-kiwi, burnt sugar, sweet, supersweet, sweet & sour, tallow, tamarind, tangerine-lime, tangerine, tea, tequila type, thyme, toffee, triple sec, tropical fruit mix, turkey, tutti frutti, vanilla, vanilla cream, vanilla custard, french vanilla, vegetable, vermouth, vinegar, balsamic vinegar, watermelon, whiskey, wildberry, wine, winter green, and yogurt. Other examples of flavors are found in 21 C.F.R. §§ 172.510, 172.515, 172.520, 172.530, 172.535, 172.575, 172.580 and 172.585, which are hereby fully incorporated by reference. A variety of food grade flavors are commercially available from Sensient Flavors Inc. in Indianapolis, Ind., Givaudan SA in Cincinnati, Ohio, and International Flavors & Fragrance in New York, N.Y.

The ratio of protein to polyanionic polymer may be varied depending on the polymer chosen. Optimal heat stability is observed when the protein content is equal to or greater than about 50% (by weight) of the protein/polyanionic complex. Optionally, the microcapsule composition may contain protein and polyanionic polymer in about a 4:1 ratio of protein to polymer.

The amount of protein (by weight) in the microcapsule composition may be from about 16.5% to about 33%, particularly from about 22% to about 28%, and more particularly from about 25% to about 27%.

The amount of polyanionic polymer (by weight) in the microcapsule composition may be from about 0.4% to about 16.5%, particularly from about 4% to about 10%, and more particularly from about 6% to about 7%.

The amount of active ingredient (by weight) in the microcapsule composition may be from about 10% to about 90%, particularly from about 50% to about 70%, and more particularly from about 64% to about 67%.

The amount of cross-linking reagent (by weight) in the microcapsule composition may be from about 0.05% to about 1%, particularly from about 0.15% to about 0.35%, based on the weight of the protein. The amount of cross-linking reagent (by weight) in the microcapsule composition is dependent on which reagent is used. For example, glutaraldehyde when used, may be from about 0.05% to about 0.45% of the protein content (by weight), particularly from about 0.15% to about 0.35% of the protein content (by weight), and more particularly from about 0.23% to about 0.27% of the protein content (by weight).

The amount of taste masking agent (by weight) in the microcapsule composition may be from about 0.1% to about 15%, particularly about 1% to about 5%. The amount of taste masking agent (by weight) in the microcapsule composition is dependent on which agent is used. For example, orange oil when used, may be from about 0.5% to about 4%, particularly from about 1% to about 3%, and more particularly from about 1.6% to about 2.2%.

The microcapsules may be formed by encapsulation techniques including, but not limited to, complex coacervation, spray drying, spray chilling, fluid bed coating, pan coating, co-extrusion, microspheres, liposomes, spinning disk, and hot melt extrusion. Complex coacervation is an encapsulation method that involves the coalescing of two polymers around a hydrophobic core, and phasing out the polymers to form a complex coating.

To prepare an aqueous dispersion of encapsulated active ingredient in one embodiment of the present invention, the active ingredient is weighed and placed in an oven and warmed to about 50-80° C., preferably about 60-70° C. Water is added to two beakers that contain magnetic stir bars. The beakers are placed on a heated stir plate and the water in each beaker is heated to about 70-90° C., preferably about 80-90° C. The desired amount of protein is weighed and slowly added to one beaker under agitation and allowed to dissolve. The desired amount of polyanionic polymer is weighed and slowly added to the other beaker under agitation and allowed to dissolve.

The taste-masking agent is weighed and added to the warmed active ingredient. This mixture is then added to the protein solution and homogenized with a hand held unit (Polytron PT2300) to create an emulsion. The emulsion is transferred to another beaker that had been placed in an empty water bath. The emulsion is mixed with an overhead mixer, and the polyanionic polymer solution is then added and mixed.

While the emulsion is still greater than about 70° C., the pH is adjusted with an acid to a pH of about 3.0 to about 4.5, more particularly about 4.0 to about 4.3. Without being bound by theory, this transitions the isoelectric point of the protein causing an electrostatic complex to form with the polyanionic polymer. Additionally, this causes the polymers to coalesce around the active ingredient in an emulsion.

Following the pH adjustment, water at room temperature is slowly added to achieve the desired total volume. Ice is then added around the outside of the beaker in the water bath and the mixture is allowed to cool under constant agitation.

As the material cools, the viscosity increases and the agitation is adjusted to prevent massive agglomeration of the individual encapsulates as they form. The material is cooled to about 5-10° C. and held for about 1 hour. The material can then be stored.

In addition, although cross-linking is not required, it may be used in the process described above. The desired amount of cross-linking reagent is added after the material is cooled to about 5-10° C. and held for about 1 hour. The material is then agitated for an additional hour at approximately 5-10° C.

The microcapsule compositions comprise an encapsulate encapsulating an active ingredient, and may have excellent heat stability properties. The microcapsule composition may help reduce or prevent breaking of the microcapsules when heated in an aqueous system. In some embodiments, the encapsulate may include a coacervate. The microcapsules may possess stability and structural integrity for at least about 60 minutes in retort processing at 121° C. and 15 PSI or hot fill pasteurization at 104° C. In other words, when the microcapsule compositions are exposed to retort processing at 121° C. and 15 PSI for 60 minutes or hot fill pasteurization at 104° C., substantially all of the active ingredient remains in the encapsulate of the microcapsule composition. As used herein, this means a microcapsule “survives” retort processing or hot fill pasteurization at these conditions. Retort processing is described in 21 C.F.R. § 113 (thermally processed low-acid foods packaged in hermetically sealed containers). Hot fill pasteurization is described in 21 C.F.R. § 114 (acidified foods) and 21 C.F.R. § 131 (milk and cream). In this manner, the microcapsule composition also provides protection for active ingredients that are sensitive to oxidation or deterioration, such as omega-3 oils. The microcapsule composition may therefore have excellent oxidative stability properties.

The microcapsule compositions of the present invention may provide the ability to include in products active ingredients that may be sensitive to oxidation or may negatively interact with other components in complex mixtures. An active ingredient encapsulated in this manner may be included in product formulations where processing parameters may otherwise damage or destroy the functionality of the unencapsulated compounds. Accordingly, the benefits associated with the active ingredients may be imparted to the end product. This may be done in a manner in which the active ingredient is able to survive conditions that it would otherwise be unable to survive without the encapsulation. Additionally, it may be done in a manner without the development of offensive flavors and odors.

Products in which the microcapsule compositions may be incorporated include, but are not limited to, food applications, beverage applications, pet food applications, pharmaceutical applications, drug delivery systems, or combinations thereof. Specific examples of products suitable for use with the composition of the present invention include all types of foods, including, but not limited to, pigmented sugar coatings and shellac coatings (alcoholic and aqueous), coatings containing oils and waxes, gum Arabic and cellulose types (e.g. HPMC-hydroxypropylmethyl cellulose), confectionery, confectionery items, cake decorations, compressed tablets, compressed products, pan-coated products, chewing gums, gum products, dragees, fondant products, marzipan products, filling compositions, cocoa icings and fat icings, chocolate and chocolate-containing products, cocoa gum, tempered chocolates, ice cream, cereals, snack products, coating compositions, glazes, cake glazes, cake bases, produce, scattered sugar decorations, nonpareils, gateaux presentation plates, sugar crystals, dextrose crystals, jam, jelly, gel and gelatin products, sweets, candy, licorice, frostings and icings, candyfloss, fat, sugar and baker's cream compositions, blancmange, puddings, desserts, flan glazing, pretzels, cookies of all types and other baked goods such as ice cream cones, crackers, biscuits, enrobed cookies, jelly beans, soft panned items, gumballs, Jordan almonds, various panned confectionery items, chocolate panned nuts, white confectionery coating/yogurt coated products like raisins, caramel pieces, malt balls, smooth hard candies including deposited types (including lozenges), gummy bears or other shapes, molded and enrobed chocolates, soups, soup mixes, cold sweet soups, sodas and carbonated drinks, beverages, alcoholic beverages, non-alcoholic beverages, dry beverage powders, beverages containing stabilizing additives (such as carboxy methyl cellulose, acidified and non-acidified milk products such as quark, yogurt, fruit prep for yogurt, cheese, cheese rings, sausage casings, etc.), dairy products, milk, powdered milk, taffy, marshmallows, baked goods, baking mixes, breakfast cereals (including ready-to-eat, instant, and hot), dairy product analogs, nondairy milk, nondairy creamers, nondairy toppings, dressings for salads, food grade inks, decorations, sprinkles, fruit and water ices, frozen confections, gelatin desserts and products, pie fillings, chips, novelty snacks, juices, orange juice, vegetable juice, sauces, spreads, pastas, meat products, fish products, frozen dairy products, cheese products, egg products, condiments, nut products, plant protein products, poultry products, fruit juices, granulated sugar (brown or white), gravy, syrup, nutritional bars, bread, tortillas, sausage, chicken, beef, fish, seafood, potato products, chips, vegetable products, fruit products, rice bran, rolls, fruit pies, cakes, brownies, scones, popcorn, oatmeal/farina, snacks, pie shells, fruit fillings, pressed snacks, rice cakes, fruit leather, peanut butter, and combinations thereof.

### EXAMPLES

Exemplary embodiments of the present heat stable microcapsules are provided in the following examples. The following examples are presented to illustrate the present microcapsules and methods for applying the microcapsules to edible substrates and to assist one of ordinary skill in making and using the same. The examples are not intended in any way to otherwise limit the scope of the invention.

### Example 1

| Component | Supplier | Amount (in grams) |
|---|---|---|
| Soy Protein Isolate | Solae | 26.7 g |
| (Supro EX 38) | St. Louis, MO | |
| Gellan Gum | Kelco | 6.7 g |
| (Kelcogel) | Chicago, IL | |
| Omega-3 Oil | Denomega | 66.7 g |
| | Boulder, CO | |

To prepare a 1000 mL batch of 10% aqueous dispersion of encapsulated oil, 66.7 grams of omega-3 oil were weighed and placed in an oven and warmed to approximately 70° C. In each of two 600 mL beakers, 300 mL of water was added. Magnetic stir bars were added to each beaker, each beaker was covered with a watch glass, and the beakers were then placed on a heated stir plate. The water in each beaker was heated to approximately 80-90° C. To one beaker, 26.7 grams of soy protein isolate (Supro EX 38 from Solae) were slowly added under agitation and allowed to dissolve. To the other beaker, 6.7 grams of gellan (Kelcogel from Kelco) were slowly added under agitation and allowed to dissolve.

When the polymers were fully dissolved, the warmed omega-3 oil was mixed into the soy protein isolate solution and homogenized with a hand-held unit (Polytron PT2300). This mixture was transferred to a 2 L beaker and agitated with an overhead mixer. The gellan solution was added and mixed well.

The pH of the mixture was adjusted to 4.4 with glacial acetic acid and the mixture was cooled to approximately 10° C. in an ice bath with agitation. The mixture was then diluted to 1000 mL with water, added slowly.

When the temperature reached approximately 5-10° C., agitation continued for 2 hours. The product was then stored under refrigeration.

To test the stability of the encapsulate through retort conditions (121° C. at 15 PSI), 80 grams of the slurry were added to each of 2 aluminum retort cans (211×300 mm, Freund Container company, Chicago, Ill.) and filled with water at 80° C. The cans were sealed using a benchtop can sealer (Dixie Canning Company, Athens, Ga.) and placed in a pilot scale retort (Dixie Canning Company, Athens, Ga.) and processed at 121° C. and 15 PSI for 55 minutes. Following the retort process, the cans were opened. The excess water was removed and the microcapsules were observed under a microscope. The microcapsules appeared intact. It was also observed that there was no free oil on the surface of the water, indicating no oil was released during retort.

To test the stability of the encapsulate through hot fill conditions (104° C. at 15 PSI), the slurry was diluted to 5% solids content and thermally processed (hot fill) using a MicroThermics pilot scale thermal processing unit. The mixture was processed with a flow of 500 mls per minute and configured for a 60 second retention time at 104° C. The product temperature at the fill spout was 82° C. and was captured in 250 ml media bottles. Once filled, the bottles were sealed with airtight screw cap lids and held upside down for 3 minutes to sterilize the lids. Following this the bottles were cooled by submerging in a tank of ambient tap water. Following the hot fill process, the bottles were opened. The excess water was removed and the microcapsules were observed under a microscope. The microcapsules appeared intact. It was also observed that there was no free oil on the surface of the water, indicating no oil was released during the process.

### Example 2

| | | Amount | |
|---|---|---|---|
| Component | Supplier | (in grams) | |
| Soy Protein Isolate | Solae | 26.3 | g |
| (Supro EX 38) | St. Louis, MO | | |
| Gellan Gum | Kelco | 6.6 | g |
| (Kelcogel) | Chicago, IL | | |
| Omega-3 Oil | Denomega | 65.8 | |
| | Boulder, CO | | |
| Glutaraldehyde (50%) | Mallinckrodt Baker | 1.3 | g |
| | Phillipsburg, NJ | | |

To prepare a 1000 mL batch of 10% aqueous dispersion of encapsulated oil, 65.8 grams of omega-3 oil were weighed and placed in an oven and warmed to approximately 70° C. In each of two 600 mL beakers, 300 mL of water was added. Magnetic stir bars were added to each beaker, each beaker was covered with a watch glass, and the beakers were then placed on a heated stir plate. The water in each beaker was heated to approximately 80-90° C. To one beaker, 26.3 grams of soy protein isolate (Supro EX 38 from Solae) were slowly added under agitation and allowed to dissolve. To the other beaker, 6.6 grams of gellan (Kelcogel from Kelco) were slowly added under agitation and allowed to dissolve.

When the polymers were fully dissolved, the warmed omega-3 oil was mixed into the soy protein isolate solution and homogenized with a hand-held unit (Polytron PT2300). This mixture was transferred to a 2 L beaker and agitated with an overhead mixer. The gellan solution was added and mixed well.

The pH of the mixture was adjusted to 4.4 with glacial acetic acid and the mixture was cooled to approximately 10° C. in an ice bath with agitation. The mixture was then diluted to 1000 mls with water, added slowly.

When the temperature reached approximately 5-10° C., agitation was continued for 1 hour. At that point, 1.3 mls of the 50% glutaraldehyde solution was added to crosslink the protein and agitation continued for 1 hour. The product was then stored under refrigeration.

To test the stability of the encapsulate through retort conditions (121° C. at 15 PSI), 80 grams of the slurry were added to each of 2 aluminum retort cans (211×300 mm, Freund Container company, Chicago, Ill.) and filled with water at 80° C. The cans were sealed using a benchtop can sealer (Dixie Canning Company, Athens, Ga.) and placed in a pilot scale retort (Dixie Canning Company, Athens, Ga.) and processed at 121° C. and 15 PSI for 55 minutes. Following the retort process, the cans were opened. The excess water was removed and the microcapsules were observed under a microscope. The microcapsules appeared intact. It was also observed that there was no free oil on the surface of the water, indicating no oil was released during retort.

To test the stability of the encapsulate through hot fill conditions (104° C. at 15 PSI), the slurry was diluted to 5% solids content and thermally processed (hot fill) using a MicroThermics pilot scale thermal processing unit. The mixture was processed with a flow of 500 mls per minute and configured for a 60 second retention time at 104° C. The product temperature at the fill spout was 82° C. and was captured in 250 ml media bottles. Once filled, the bottles were sealed with airtight screw cap lids and held upside down for 3 minutes to sterilize the lids. Following this the bottles were cooled by submerging in a tank of ambient tap water. Following the hot fill process, the bottles were opened. The excess water was removed and the microcapsules were observed under a microscope. The microcapsules appeared intact. It was also observed that there was no free oil on the surface of the water, indicating no oil was released during the process.

### Example 3

| Component | Supplier | Amount (in grams) |
|---|---|---|
| Soy Protein Isolate | Solae | 25.8 g |
| (Supro EX 38) | St. Louis, MO | |
| Gellan Gum | Kelco | 6.5 g |
| (Kelcogel) | Chicago, IL | |
| Omega-3 Oil | Denomega | 64.5 g |
| | Boulder, CO | |
| Orange Flavor | Sensient Flavors | 1.9 g |
| | Indianapolis, IN | |

To prepare a 1000 mL batch of 10% aqueous dispersion of encapsulated oil, 64.5 grams of omega-3 oil were weighed and placed in an oven and warmed to approximately 60-70° C. In each of two 600 mL beakers, 300 mL of water was added. Magnetic stir bars were added to each beaker, and the beakers were then placed on a heated stir plate. The water in each beaker was heated to approximately 80-90° C. To one beaker, 25.8 grams of the soy protein isolate (Supro EX 38 from Solae) were slowly added under agitation and allowed to dissolve. To the other beaker, 6.5 grams of the gellan (Kelcogel from Kelco) were slowly added under agitation and allowed to dissolve.

1.9 grams of the orange flavor (oil soluble) were added to the warmed oil. This mixture was then added to the soy protein isolate solution and homogenized with a hand-held unit (Polytron PT2300) to create an emulsion. The emulsion was transferred to a 2000 mL beaker that had been placed in an empty water bath. The emulsion was then mixed with an overhead mixer, and the gellan solution was added and allowed to mix.

While the emulsion was still above 70° C., the pH was adjusted with glacial acetic acid to a pH of about 4.0 to about 4.5. Without being limited by theory, this transitions the isoelectric point of the protein causing an electrostatic complex to form with the carbohydrate polymer. Additionally, this causes the polymers to coalesce around the omega-3 oil droplets in an emulsion.

Following the pH adjustment, water at room temperature was slowly added for a total volume of 1000 mL. Ice was then added around the outside of the beaker in the water bath and the mixture was allowed to cool under constant agitation.

As the material cooled, the viscosity increased and the agitation was adjusted to prevent massive agglomeration of the individual encapsulates as they formed. The material was cooled to approximately 5-10° C. and held for 1 hour.

To test the stability of the encapsulate through retort conditions (121° C. at 15 PSI), 80 grams of the slurry were added to each of 2 aluminum retort cans (211×300 mm, Freund Container company, Chicago, Ill.) and filled with water at 80° C. The cans were sealed using a benchtop can sealer (Dixie Canning Company, Athens, Ga.) and placed in a pilot scale retort (Dixie Canning Company, Athens, Ga.) and processed at 121° C. and 15 PSI for 55 minutes. Following the retort process, the cans were opened. The excess water was removed and the microcapsules were observed under a microscope. The microcapsules appeared intact. It was also observed that there was no free oil on the surface of the water, indicating no oil was released during retort.

To test the stability of the encapsulate through hot fill conditions (104° C. at 15 PSI), the slurry was diluted to 5% solids content and thermally processed (hot fill) using a MicroThermics pilot scale thermal processing unit. The mixture was processed with a flow of 500 mls per minute and configured for a 60 second retention time at 104° C. The product temperature at the fill spout was 82° C. and was captured in 250 ml media bottles. Once filled, the bottles were sealed with airtight screw cap lids and held upside down for 3 minutes to sterilize the lids. Following this the bottles were cooled by submerging in a tank of ambient tap water. Following the hot fill process, the bottles were opened. The excess water was removed and the microcapsules were observed under a microscope. The microcapsules appeared intact. It was also observed that there was no free oil on the surface of the water, indicating no oil was released during the process.

### Example 4

| Component | Supplier | Amount (in grams) |
|---|---|---|
| Soy Protein Isolate | Solae | 25.8 g |
| (Supro EX 38) | St. Louis, MO | |
| Gellan Gum | Kelco | 6.5 g |
| (Kelcogel) | Chicago, IL | |
| Omega 3 Oil | Denomega | 64.5 g |
| | Boulder, CO | |
| Orange Flavor | Solae | 1.9 g |
| | St. Louis, MO | |
| Glutaraldehyde (50%) | Mallinckrodt Baker | 1.3 g |
| | Phillipsburg, NJ | |

To prepare a 1000 mL batch of 10% aqueous dispersion of encapsulated oil, 64.5 grams of omega-3 oil were weighed and placed in an oven and warmed to approximately 60-70° C. In each of two 600 mL beakers, 300 mL of water was added. Magnetic stir bars were added to each beaker, and the beakers were then placed on a heated stir plate. The water in each beaker was heated to approximately 80-90° C. To one beaker, 25.8 grams of the soy protein isolate (Supro EX 38 from Solae) were slowly added under agitation and allowed to dissolve. To the other beaker, 6.5 grams of the gellan (Kelcogel from Kelco) were slowly added under agitation and allowed to dissolve.

1.9 grams of the orange flavor (oil soluble) were added to the warmed oil. This mixture was then added to the soy protein isolate solution and homogenized with a hand-held unit (Polytron PT2300) to create an emulsion. The emulsion was transferred to a 2000 mL beaker that had been placed in an empty water bath. The emulsion was then mixed with an overhead mixer, and the gellan solution was added and allowed to mix.

While the emulsion was still above 70° C., the pH was adjusted with glacial acetic acid to a pH of about 4.0 to about 4.5. Without being limited by theory, this transitions the isoelectric point of the protein causing an electrostatic complex to form with the carbohydrate polymer. Additionally, this causes the polymers to coalesce around the omega-3 oil droplets in an emulsion.

Following the pH adjustment, water at room temperature was slowly added for a total volume of 1000 mL. Ice was then added around the outside of the beaker in the water bath and the mixture was allowed to cool under constant agitation.

As the material cooled, the viscosity increased and the agitation was adjusted to prevent massive agglomeration of the individual encapsulates as they formed. The material was cooled to approximately 5-10° C. and held for 1 hour. At that point, 1.3 mls of the 50% glutaraldehyde solution was added to crosslink the protein and agitation continued for 1 hour. The product was then stored under refrigeration.

To test the stability of the mixture through retort conditions (121° C. at 15 PSI), 80 grams of the slurry were added to each of 2 aluminum retort cans (211×300 mm, Freund Container company, Chicago, Ill.) and filled with water at 80° C. The cans were sealed using a benchtop can sealer (Dixie Canning Company, Athens, Ga.) and placed in a pilot scale retort (Dixie Canning Company, Athens, Ga.) and processed at 121° C. and 15 PSI for 55 minutes. Following the retort process, the cans were opened. During the heat, the microcapsules appeared to have agglomerated to visible particle size and sealed on the bottom. The excess water was removed and the microcapsules were observed under a microscope. The microcapsules appeared intact. It was also observed that there was no free oil on the surface of the water, indicating no oil was released during retort.

To test the stability of the encapsulate through hot fill conditions (104° C. at 15 PSI), the slurry was diluted to 5% solids content and thermally processed (hot fill) using a MicroThermics pilot scale thermal processing unit. The mixture was processed with a flow of 500 mls per minute and configured for a 60 second retention time at 104° C. The product temperature at the fill spout was 82° C. and was captured in 250 ml media bottles. Once filled, the bottles were sealed with airtight screw cap lids and held upside down for 3 minutes to sterilize the lids. Following this the bottles were cooled by submerging in a tank of ambient tap water. Following the hot fill process, the bottles were opened. The excess water was removed and the microcapsules were observed under a microscope. The microcapsules appeared intact. It was also observed that there was no free oil on the surface of the water, indicating no oil was released during the process.

### Example 5

To test the stability of encapsulated fish oil through retort processing, a virgin salmon oil was obtained from Marine Harvest Ingredients and used as the substrate for encapsulation. Two batches were prepared in the lab by complex coacervation, batch one prepared as described in Example 1, and the second batch being crosslinked with glutaraldehyde as in Example 2.

The final oil content of the finished encapsulated products was approximately 64-65%. The coacervated oil was left in a slurry form of approximately 10% solids.

80 grams of the slurry was added to each of 5 aluminum cans (211×300 mm, Freund Container company, Chicago, Ill.) and filled with water at 80° C. For a control, 5 grams of the raw oil of each type was added to each of 5 cans and likewise filled with water. The cans were sealed using a benchtop can sealer (Dixie Canning Company, Athens, Ga.) and sets of 3 of each formula were retorted for 60 minutes at 121° C. and 15 PSI using a pilot scale retort chamber (Dixie Canning Company, Athens, Ga.).

To test whether encapsulating these oxidatively sensitive oils increased the shelf stability following retorting, the retorted cans containing the non-encapsulated or the encapsulated fish oil were placed in an environmental chamber set at 40° C. for accelerated storage. Additionally, to determine the shelf life of the encapsulate slurry, samples of the oil and of each encapsulate type were placed in 6 one ounce amber glass jars and placed in the 40° C. chamber. Samples were analyzed for DHA content after 0, 3, 6, 9 and 12 weeks storage. For an edible oil, it is generally considered that 1 week at 40° C. is equivalent to approximately 2 months ambient shelf life.

To extract the oil from the encapsulate, samples were concentrated by removing as much water as possible by filtration. The encapsulates were then dispersed in a chloroform/methanol mixture (1:2) and the capsules were ruptured to release the oil by high shear homogenation using a hand held Ultra Turex homogenizer. A potassium chloride solution was added to break the emulsion, and the chloroform layer removed. The chloroform was then evaporated under a flow of nitrogen gas. A 1 gram sample of the extracted oil was removed and analyzed for peroxide value following AOCS method Cd 8b-90.

Peroxide values (PV) can provide information on the level of oxidation during the initial stage of the reaction. In the tables below, the PVs of these oils through retort and storage are presented.

| Sample | 0 wks | 3 wks | 6 wks | 9 wks | 12 wks |
|---|---|---|---|---|---|
| Oil | 4.3 | 12.8 | 98.5 | 195.3 | 248.0 |
| Oil (retorted) | 9.4 | 24.6 | 32.9 | 39.62 | 52.6 |
| Oil encap (retorted) | 5.3 | 7.2 | 9.3 | 9.9 | 12.3 |
| Oil encap XL* (retorted) | 4.8 | 5.6 | 6.0 | 6.5 | 8.32 |

*XL = cross-linked

As can be seen above, the PV of the raw oil was significantly higher than those of the retorted samples after accelerated storage. The process of filling the cans at an elevated temperature and leaving a very limited headspace in the can results in much of the available oxygen being purged prior to the can being sealed. This process significantly limits the oxygen available to oxidize the fatty acids during retort and storage. As a consequence, both the raw and encapsulated oils show a much lower PV over time as compared to the non-retorted raw oils. However, the retorted non-encapsulated oils had a significantly higher PV and a much stronger odor upon opening the can than did the encapsulated oil, indicating that oxidation had been initiated to a higher degree in the raw oils than in the encapsulated oils. There was no difference in stability between the crosslinked and the non-crosslinked encapsulated oils.

While the retort process can have an oxygen limiting effect, oxidation of long chain unsaturated fatty acids is initiated. Following retort and storage, this oxidation can cause flavor and odor problems to the finished product as well as reduces the stability of the product once the retort container is opened. Encapsulation of these oils can significantly reduce the flavor and odor issues and will help insure that the omega-3 content remains in the product during shelf life.

### Example 6

In order to test and compare the structural stability through retort processing of encapsulated oil formulations, the following series of encapsulates were prepared in the lab:

EX 38/Gellan + XL

| | | Amount | |
|---|---|---|---|
| Component | Supplier | (in grams) | |
| Soy Protein Isolate | Solae | 26.3 | g |
| (Supro EX 38) | St. Louis, MO | | |
| Gellan Gum | Kelco | 6.6 | g |
| (Kelcogel) | Chicago, IL | | |
| Omega-3 Oil | Denomega | 65.8 | |
| | Boulder, CO | | |
| Glutaraldehyde (50%) | Mallinckrodt Baker | 1.3 | g |
| | Phillipsburg, NJ | | |

EX 38/Gellan

| Component | Supplier | Amount (in grams) |
|---|---|---|
| Soy Protein Isolate | Solae | 26.7 g |
| (Supro EX 38) | St. Louis, MO | |
| Gellan Gum | Kelco | 6.7 g |
| (Kelcogel) | Chicago, IL | |
| Omega-3 Oil | Denomega | 66.7 g |
| | Boulder, CO | |

SP 248/Gellan + XL

| | | Amount | |
|---|---|---|---|
| Component | Supplier | (in grams) | |
| Soy Protein Isolate | Solae | 26.3 | g |
| (Supro SP 248) | St. Louis, MO | | |
| Gellan Gum | Kelco | 6.6 | g |
| (Kelcogel) | Chicago, IL | | |
| Omega-3 Oil | Denomega | 65.8 | |
| | Boulder, CO | | |
| Glutaraldehyde (50%) | Mallinckrodt Baker | 1.3 | g |
| | Phillipsburg, NJ | | |

SP 248/Gellan

| Component | Supplier | Amount (in grams) |
|---|---|---|
| Soy Protein Isolate | Solae | 26.7 g |
| (Supro SP 248) | St. Louis, MO | |
| Gellan Gum | Kelco | 6.7 g |
| (Kelcogel) | Chicago, IL | |
| Omega-3 Oil | Denomega | 66.7 g |
| | Boulder, CO | |

XT 219/Gellan + XL

| | | Amount | |
|---|---|---|---|
| Component | Supplier | (in grams) | |
| Soy Protein Isolate | Solae | 26.3 | g |
| (Supro XT 219) | St. Louis, MO | | |
| Gellan Gum | Kelco | 6.6 | g |
| (Kelcogel) | Chicago, IL | | |
| Omega-3 Oil | Denomega | 65.8 | |
| | Boulder, CO | | |
| Glutaraldehyde (50%) | Mallinckrodt Baker | 1.3 | g |
| | Phillipsburg, NJ | | |

XT 219/Gellan

| Component | Supplier | Amount (in grams) |
|---|---|---|
| Soy Protein Isolate | Solae | 26.7 g |
| (Supro XT 219) | St. Louis, MO | |
| Gellan Gum | Kelco | 6.7 g |
| (Kelcogel) | Chicago, IL | |
| Omega-3 Oil | Denomega | 66.7 g |
| | Boulder, CO | |

Gelatin/Gum Arabic + XL

| | | Amount | |
|---|---|---|---|
| Component | Supplier | (in grams) | |
| Gelatin, Type B | Great Lakes Gelatin | 16.5 | g |
| (250 Bloom) | Grayslake, IL | | |
| Gum Arabic | Tic Gums | 16.5 | g |
| (Spray dry powder) | Belcamp, MD | | |
| Omega-3 Oil | Denomega | 66.1 | |
| | Boulder, CO | | |
| Glutaraldehyde (50%) | Mallinckrodt Baker | 0.8 | g |
| | Phillipsburg, NJ | | |

Gelatin/Gum Arabic

| Component | Supplier | Amount (in grams) |
|---|---|---|
| Gelatin, Type B | Great Lakes Gelatin | 16.7 g |
| (250 Bloom) | Grayslake, IL | |
| Gum Arabic | Tic Gums | 16.7 g |
| (Spray dry powder) | Belcamp, MD | |
| Omega-3 Oil | Denomega | 66.7 g |
| | Boulder, CO | |

Fish Gelatin/Gum Arabic + XL

| | | Amount | |
|---|---|---|---|
| Component | Supplier | (in grams) | |
| Fish Gelatin | Food Industry | 16.5 | g |
| (240 Bloom) | Technology | | |
| | Miami Beach, FL | | |
| Gum Arabic | Tic Gums | 16.5 | g |
| (Spray dry powder) | Belcamp, MD | | |
| Omega-3 Oil | Denomega | 66.1 | |
| | Boulder, CO | | |
| Glutaraldehyde (50%) | Mallinckrodt Baker | 0.8 | g |
| | Phillipsburg, NJ | | |

Fish Gelatin/Gum Arabic

| Component | Supplier | Amount (in grams) |
|---|---|---|
| Fish Gelatin | Food Industry Technology | 16.7 g |
| (240 Bloom) | Miami Beach, FL | |
| Gum Arabic | Tic Gums | 16.7 g |
| (Spray dry powder) | Belcamp, MD | |
| Omega-3 Oil | Denomega | 66.7 g |
| | Boulder, CO | |

EX 38/Gum Arabic + XL

| | | Amount | |
|---|---|---|---|
| Component | Supplier | (in grams) | |
| Soy Protein Isolate | Solae | 16.5 | g |
| (Supro EX 38) | St. Louis, MO | | |
| Gum Arabic | Tic Gums | 16.5 | g |
| (Spray dry powder) | Belcamp, MD | | |
| Omega-3 Oil | Denomega | 66.1 | |
| | Boulder, CO | | |
| Glutaraldehyde (50%) | Mallinckrodt Baker | 0.8 | g |
| | Phullipsburg, NJ | | |

EX 38/Gum Arabic

| Component | Supplier | Amount (in grams) |
|---|---|---|
| Soy Protein Isolate | Solae | 16.7 g |
| (Supro EX 38) | St. Louis, MO | |
| Gum Arabic | Tic Gums | 16.7 g |
| (Spray dry powder) | Belcamp, MD | |
| Omega-3 Oil | Denomega | 66.7 g |
| | Boulder, CO | |

Gelatin/Gellan + XL

| | | Amount | |
|---|---|---|---|
| Component | Supplier | (in grams) | |
| Gelatin, Type B | Great Lakes Gelatin | 26.3 | g |
| (250 Bloom) | Grayslake, IL | | |
| Gellan Gum | Kelco | 6.6 | g |
| (Kelcogel) | Chicago, IL | | |
| Omega-3 Oil | Denomega | 65.8 | |
| | Boulder, CO | | |
| Glutaraldehyde (50%) | Mallinckrodt Baker | 1.3 | g |
| | Phillipsburg, NJ | | |

Gelatin/Gellan

| Component | Supplier | Amount (in grams) |
|---|---|---|
| Gelatin, Type B | Great Lakes Gelatin | 26.7 g |
| (250 Bloom) | Grayslake, IL | |
| Gellan Gum | Kelco | 6.7 g |
| (Kelcogel) | Chicago, IL | |
| Omega-3 Oil | Denomega | 66.7 g |
| | Boulder, CO | |

Samples of the encapsulation formulas described above were prepared by the methods listed in the table below.

| Sample | Encapsulation procedure |
|---|---|
| EX 38/Gellan + XL* | As described in Example 2 |
| EX 38/Gellan | As described in Example 1 |
| SP 248/Gellan + XL | As described in Example 2 |
| SP 248/Gellan | As described in Example 1 |
| XT 219/Gellan + XL | As described in Example 2 |
| XT 219/Gellan | As described in Example 1 |
| Gelatin/Gum Arabic + XL | As described below |
| Gelatin/Gum Arabic | As described below |
| Fish Gelatin/Gum Arabic + XL | As described below |
| Fish Gelatin/Gum Arabic | As described below |
| EX 38/Gum Arabic + XL | As described below |
| EX 38/Gum Arabic | As described below |
| Gelatin/Gellan + XL | As described in Example 2 |
| Gelatin/Gellan | As described in Example 1 |

### Preparation Method for Formulas Using Gum Arabic

To prepare a 1000 ml batch of 10% aqueous dispersion of encapsulated oil, the appropriate grams of omega-3 oil were weighed and placed in an oven and warmed to approximately 70° C. In each of two 600 ml beakers, 300 ml of water was added. Magnetic stir bars were added to each beaker, each beaker was covered with a watch glass, and the beakers were then placed on a heated stir plate. The water in each beaker was heated to approximately 60-97° C. To one beaker, the stated grams of the protein polymer were slowly added under agitation and allowed to dissolve. To the other beaker, the stated grams of Gum Arabic (Tic Gums) were slowly added under agitation and allowed to dissolve.

When the polymers were fully dissolved, the warmed omega-3 oil was mixed into the protein solution and homogenized with an Ultra Turrax Homogenizer. This mixture was transferred to a 2 L beaker and agitated with an overhead mixer. The gelatin solution was added and mixed well.

The pH of the mixture was adjusted to 4.0-4.5 with glacial acetic acid and the mixture was cooled to approximately 10° C. in an ice bath with agitation. The mixture was then diluted to 1000 ml with water, added slowly.

For the formulas requiring cross-linking, when the temperature reached approximately 5-10° C., 0.8 mls 50% glutaraldehyde was added to crosslink the protein and agitation continued for 1 hour. The product was then stored under refrigeration.

### Analysis of Stability Through Retort Processing

For each encapsulation formula, 80 grams of the slurry was added to each of 12 aluminum cans (211×300 mm, Freund Container company, Chicago, Ill.) and filled with water at 80° C. The cans were sealed using a benchtop can sealer (Dixie Canning Company, Athens, Ga.) and sets of 3 of each formula were retorted for 15, 30, 45 and 60 minutes respectively at 121° C. and 15 PSI using a pilot scale retort chamber (Dixie Canning Company, Athens, Ga.)

The structural stability of the encapsulation formulas was determined by measuring the quantity of the encapsulated oil that was released from the microcapsules during the retort process by the following method.

For each sample, the can was opened using a standard kitchen variety can opener, and the contents poured into a 600 ml beaker. The can was then flushed with 20 mls hexane to remove any residual oil soluble material and the resulting mixture was added to the beaker. A magnetic stir bar was added and the can contents and hexane were allowed to mix gently for 1 minute to dissolve any oil released from the encapsulates into the hexane phase. The mixture was then filtered through glass wool to remove the encapsulation material into a 1000 ml separatory funnel and the 2 phases were allowed to separate.

Once separation was complete, the bottom aqueous layer was removed, leaving the upper hexane/oil layer. The hexane/oil mixture was collected into a 250 ml beaker that had been tared using a 4-place analytical balance. The beaker was placed in a fume hood and the hexane was allowed to completely evaporate, leaving the oil as a residue. The beaker containing the residue was weighed on the 4-place analytical balance, and the weight of the oil calculated. The percentage of oil released was then calculated by dividing the weight of the residue by the weight of the oil in the original sample and multiplying the result by 100. As stated above, the sets were analyzed in triplicate and the percent release was averaged. The results are presented in the table below.

% Release

| Sample | 15 minutes | 30 minutes | 45 minutes | 60 minutes |
|---|---|---|---|---|
| EX 38/Gellan + XL* | 0.1 | 0.1 | 0.1 | 0.1 |
| EX 38/Gellan | 0.1 | 0.1 | 0.2 | 0.1 |
| SP 248/Gellan + XL | 22.8 | 30.1 | 33.5 | 36.3 |
| SP 248/Gellan | 70.9 | 76.4 | 76.8 | 73.4 |
| XT 219/Gellan + XL | 41.7 | 46.2 | 58.9 | 58.9 |
| XT 219/Gellan | 82.6 | 84.7 | 83.3 | 88.5 |
| Gelatin/Gum Arabic + XL | 71.1 | 81.0 | 82.9 | 80.7 |
| Gelatin/Gum Arabic | 97.8 | 100.2 | 99.9 | 99.9 |
| Fish Gelatin/Gum Arabic + XL | 42.9 | 52.8 | 47.3 | 55.4 |
| Fish Gelatin/Gum Arabic | 101.9 | 99.9 | 101.0 | 99.4 |
| EX 38/Gum Arabic + XL | 1.7 | 1.9 | 1.6 | 1.7 |
| EX 38/Gum Arabic | 3.2 | 5.0 | 6.0 | 8.2 |
| Gelatin/Gellan + XL | 70.0 | 67.6 | 72.9 | 76.5 |
| Gelatin/Gellan | 98.9 | 99.4 | 99.7 | 101.5 |

*XL = crosslinked with glutaraldehyde

The encapsulation formulas containing the soy protein isolate EX 38 all had superior heat stability, with the EX 38/Gellan formulation being the optimum. It is especially important to note that in the EX 38/Gellan formulations that there was no significant difference in release between the samples that were cross-linked, and those that were not. With the EX 38/Gum Arabic formulations cross-linking did have a minor affect, but not to the same degree as is seen in the formulation that did not contain EX 38.

### Example 7

To evaluate the use of a taste-masked encapsulated omega 3 oil in a retorted food product, batches of the coacervate were made, one following the method and formula described in Example 1 (no taste masking agent), and the second following the method described in Example 3 but using the following formula:

| Component | Supplier | Amount (in grams) |
|---|---|---|
| Soy Protein Isolate | Solae | 25.8 g |
| (Supro EX 38) | St. Louis, MO | |
| Gellan Gum | Kelco | 6.5 g |
| (Kelcogel) | Chicago, IL | |
| Omega-3 Oil | Denomega | 64.5 g |
| | Boulder, CO | |
| Natural Tomato Flavor | Sensient Flavors | 1.9 g |
| | Indianapolis, IN | |

For each batch, the product was left in a slurry form of approximately 10% solids. The slurry containing the encapsulated omega-3 oil was then added, in an amount to provide 35 milligrams of omega-3 fatty acids per each 8 ounce serving, to 1 liter of a standard tomato soup that had been prepared and warmed to 88° C. The mixture was well stirred and transferred into aluminum retort cans (211×300 mm, Freund Container company, Chicago, Ill.). The cans were sealed using a benchtop can sealer (Dixie Canning Company, Athens, Ga.) and placed in a pilot scale retort (Dixie Canning Company, Athens, Ga.) and processed at 121° C. and 15 PSI for 55 minutes.

After processing, the cans were cooled and held for 7 days at ambient temperature. After this period, a can of each encapsulate formula was open and the contents transferred to a small saucepan and warmed to 85° C. The soups were tasted and the results compared. The soup containing the encapsulated omega-3 oil without the taste masking agent had a slight, but noticeable fish off-flavor. There was no fish off-flavor noted in the soup with the encapsulated omega-3 oil containing the natural tomato taste masking agent. The tasting was repeated every 7 days with fresh cans for 3 additional weeks, with the same results.

### Example 8

To evaluate the use of a taste-masked encapsulated omega-3 oil in a retorted food product, batches of the coacervate were made, one following the method and formula described in Example 2 (no taste masking agent), and the second following the method described in Example 4 but using the following formula:

| Component | Supplier | Amount (in grams) |
|---|---|---|
| Soy Protein Isolate | Solae | 25.8 g |
| (Supro EX 38) | St. Louis, MO | |
| Gellan Gum | Kelco | 6.5 g |
| (Kelcogel) | Chicago, IL | |
| Omega 3 Oil | Denomega | 64.5 g |
| | Boulder, CO | |
| Lemon Flavor | Solae | 1.9 g |
| | St. Louis, MO | |
| Glutaraldehyde | Mallinckrodt Baker | 1.3 g |
| (50%) | Phillipsburg, NJ | |

For each batch, the product was left in a slurry form of approximately 10% solids. The slurry containing the encapsulated omega-3 oil was then added, in an amount to provide 35 milligrams of omega-3 fatty acids per each 8 ounce serving, to 2 liters of a standard lemonade formula that had been prepared. The mixture was well stirred and thermally processed (hot fill) using a MicroThermics pilot scale thermal processing unit. The mixture was processed with a flow of 500 mls per minute and configured for a 60 second retention time at 104° C. The product temperature at the fill spout was 82° C. and was captured in 250 ml media bottles. Once filled, the bottles were sealed with airtight screw cap lids and held upside down for 3 minutes to sterilize the lids. Following this the bottles were cooled by submerging in a tank of ambient tap water.

After processing, the bottles were held for 2 days at refrigerator temperature. After this period, a bottle of each encapsulate formula was opened and the contents transferred to small plastic cups, tasted, and the results compared. The lemonade containing the encapsulated omega-3 oil without the taste masking agent had a slight, but noticeable fish off-flavor. There was no fish off-flavor noted in the lemonade with the encapsulated omega-3 oil containing the lemon flavor masking agent. The tasting was repeated 7 days later with fresh bottles with the same results.

What is claimed is:

1. A method of manufacturing a microcapsule, the method comprising complex coacervating a protein and a polyanionic polymer to form a coacervate, the coacervate being at least a portion of the microcapsule, and wherein a cross-linking reagent is not used during the coacervation.

2. The method of claim 1, further comprising cross-linking the microcapsule using a cross-linking reagent.

3. The method of claim 2, wherein the cross-linking reagent comprises at least one of a transglutaminase, an aldehyde, tannic acid, alum, and a combination thereof.

4. The method of claim 1, wherein the protein comprises a soy protein isolate.

5. The method of claim 1, wherein the protein comprises at least one of a milk protein, a canola protein, a whey protein, a gelatin (bovine), a gelatin (porcine), a gelatin (fish), an albumin, and a combination thereof.

6. The method of claim 1, wherein the polyanionic polymer comprises at least one of a polycarboxylated polymer, a polyaspartic acid or a salt thereof, a polyglutamic acid or a salt thereof, an agar, an alginate, a starch, gum Arabic, carrageenan, pectin, hydroxypropylmethyl cellulose, gellan gum, and a combination thereof.

7. The method of claim 1, further comprising encapsulating an active ingredient in the coacervate.

8. The method of claim 7, wherein the active ingredient comprises at least one of a pharmaceutically active drug, a catalyst, a living cell, a dead cell, a tissue, a pesticide, an herbicide, a nutrient, a fertilizer, a seed, an aquacultural feed, an aquacultural pigment, a cosmetic product, a particulate, a food ingredient, a color additive, a taste additive, a sweetener, an oil, a fatty acid, and a combination thereof.

9. The method of claim 7, wherein the active ingredient comprises a fatty acid.

10. The method of claim 7, wherein the active ingredient comprises an omega-3 fatty acid.

11. The method of claim 1, further comprising adding a taste-masking agent to the microcapsule.

12. The composition of claim 11, wherein the taste masking agent comprises at least one of a synthetic flavor, an artificial flavor, a natural flavor, and a combination thereof.

13. The method of claim 1, wherein the protein and polyanionic polymer are heated to about 70° C. to about 90° C.

14. The method of claim 7, further comprising heating the active ingredient to about 50° C. to about 80° C. prior to encapsulation.

15. The method of claim 1, wherein the microcapsule composition comprises about 16.5% to about 33% by weight protein.

16. The method of claim 1, wherein the microcapsule composition comprises about 0.4% to about 16.5% by weight polyanionic polymer.

17. The method of claim 7, wherein the microcapsule composition comprises about 10% to about 90% by weight active ingredient.

18. The method of claim 11, wherein the microcapsule composition comprises about 0.1% to about 15% by weight taste masking agent.

19. A microcapsule composition comprising a protein, a polyanionic polymer, and a taste masking agent.

20. The composition of claim 19, wherein the protein comprises a soy protein isolate.

21. An encapsulate comprising a protein and gellan gum.

22. The encapsulate of claim 21, wherein the encapsulate comprises a coacervate.

23. The coacervate of claim 22, further comprising an active ingredient and a taste masking agent.

24. The coacervate of claim 22, wherein the protein comprises soy protein isolate, and the active ingredient comprises a fatty acid.

24. A microcapsule composition comprising an encapsulate comprising a protein and a polyanionic polymer, and an active ingredient encapsulated in the encapsulate, wherein when the microcapsule is exposed to retort processing at 121° C. and 15 PSI for 60 minutes, or hot fill pasteurization at 104° C., substantially all of the active ingredient remains in the encapsulate.

25. A method of delivering an active ingredient to a subject, the method comprising incorporating a microcapsule composition comprising a protein, a polyanionic polymer, an active ingredient, and a taste masking agent into at least one of a food, a beverage, a pet food, a pharmaceutical, a drug delivery system, and a combination thereof; and
administering at least one of a food, a beverage, a pet food, a pharmaceutical, a drug delivery system, and a combination thereof to the subject.
